(19) United States
(12) Patent Application Publication
(10) Pub. No.: US 2013/0126404 A1
(43) Pub. Date: May 23, 2013

(54) Device With A Peristaltic Hose Pump And Associated Method

(71) Applicants: Soren Gronau, Nauheim (DE); Ralf Mueller, Bad Homburg (DE)

(72) Inventors: Soren Gronau, Nauheim (DE); Ralf Mueller, Bad Homburg (DE)

(73) Assignee: Fresenius Medical Care Deutschland GmbH, Bad Homburg (DE)

(21) Appl. No.: 13/813,739

(22) PCT Filed: Aug. 1, 2011
(86) PCT No.: PCT/EP2011/003850

§ 371 Date: Feb. 1, 2013

(30) Foreign Application Priority Data
Aug. 3, 2010 (DE) 10 2010 033 241.0

(51) Int. Cl. A61M 1/16; F04B 13/00

(52) U.S. Cl. CPC A61M 1/16; F04B 13/00
USPC 210/97; 417/290; 417/63; 417/53

(57) ABSTRACT

The present invention relates to an apparatus having at least one hose roller pump, wherein the hose roller pump has at least one pump bed and at least one hose or hose part inserted or insertable into the pump bed, and having at least one pressure detection means which is arranged downstream of the hose roller pump and by means of which the time extent of the pressure of a fluid conveyed by means of the hose roller pump can be determined, and having at least one evaluation means by means of which the occlusion of the hose or hose part inserted or insertable into the pump bed can be monitored with reference to the time difference and/or to the angular difference between at least one maximum and at least one minimum of the determined time extent of the pressure and/or with reference to a local minimum of the determined time extent of the pressure. The invention furthermore in particular relates to a method therefor.

The present invention relates to an apparatus having a hose roller pump and to a method therefor.

In dialysis, hose roller pumps are generally used for conveying fluids such as substituates or blood. The rollers of these peristaltic pumps are spring-loaded to limit the maximum pressure built up in the event of an error. In normal operation, it is assumed that complete occlusion is present during the engagement of a roller. Whether this is the case or not is not subject to any constant monitoring. The function of the rotor is only checked ex works.

The prefilter pressure (pressure between the blood pump and the dialyzer) typically increases in the course of the dialysis. The flow resistance of the dialyzer increases e.g. due to the closure of individual capillaries. The flow of hose roller pumps is normally not dependent on the counterpressure. However, if the counterpressure exceeds a limit value, the roller then currently in engagement can briefly lift off. A loss of the occlusion results in the reduction of the actual blood flow; however, it is more important that a rotor can result in increased hemolysis in this state.

Approaches are already known from the prior art to monitor the working behavior of a hose roller pump and also approaches to recognize stenoses in connection with an extracorporeal blood circuit.

DE 10 2006 011 346 A1 relates to a method and to an apparatus for operating an electric peristaltic hose pump, with a check being made by means of an evaluation of the electric power consumption of the hose roller pump whether a complete occlusion or only a partial occlusion of the hose roller pump is present.

EP 1 245 242 A2 relates to a method and to an apparatus for recognizing stenoses in a hose line system, with the hose line system having a hose roller pump for conveying a fluid. In this respect, the oscillating pressure is evaluated upstream of the pump and a presumption of a stenosis is made on the basis of a change in the pressure signal.

DE 199 01 078 C1 describes an apparatus for recognizing stenoses in extracorporeal blood treatment. For this purpose, the pressure signal received by means of the arterial pressure sensor is analyzed. In this respect, an oscillating pressure signal is generated by the pressure pump which propagates over the hose line system. In the event of a sufficiently strong stenosis between the blood pump and the blood treatment unit, the pressure after the blood pump increases so much that the infusion of the pump rollers is partially cancelled. This has the consequence that some blood flows into the line system before the blood pump at times in the pumping procedure, which results in an enlargement of the pulse amplitude measured at the arterial pressure sensor. At the same time, the generally negative center pressure moves in the direction of the zero line since the conveyed quantity decreases. A stenosis can correspondingly be assumed.

A method is known from WO 97/10013 for recognizing a stenosis at the patient access in which an oscillating pressure signal is analyzed which is transferred via the hose line system. The pump signal due to the rotation of the blood pump is extracted from the pressure signal to be able to detect the pulsed signal of the patient. To the extent that the pulsed signal of the patient cannot be detected, a stenosis is assumed in the extracorporeal blood circuit or subsequent to the extracorporeal blood circuit.

It is the object of the present invention to further develop an apparatus and a method of the initially named kind in an advantageous manner, in particular such that possible blood damage by an incompletely occluding rotor of a hose roller pump can be recognizable as immediately as possible and an improvement of the operating safety of a hose roller pump can be achieved.

This object is solved in accordance with the invention by an apparatus having the features of claim 1. Provision is accordingly made that an apparatus is provided with at least one hose roller pump, wherein the hose roller pump has at least one pump bed and at least one hose or hose part inserted or insertable into the pump bed, and with at least one pressure detection means which is arranged downstream of the hose roller pump and by means of which the time extent of the pressure of a fluid conveyed by means of the hose roller pump can be determined, and with at least one monitoring means by means of which the occlusion of the hose or hose part inserted or insertable into the pump bed can be monitored with reference to the time and/or angular difference between at least one maximum and at least one minimum of the determined time extent of the pressure and/or with reference to a local minimum of the determined time extent of the pressure.

The angular difference is in particular the difference of the angular positions of the rotor of the hose roller pump between the maxima and the minima of the pressure. The time difference and the angular difference can correlate with one another since both can depend on the rotational movement of the rotor.

The monitoring means can, for example, be formed by a control and/or regulation unit such as by a corresponding controller or microcomputer. It is generally also conceivable that, in a case in which the apparatus cooperates with a larger or with another machine, the function of the monitoring means is taken over by a part of the machine control of the machine.

It is particularly advantageous that now a particular simple monitoring of the occlusion of a hose roller pump is possible with reference to the time extent of the pressure maxima with respect to the pressure minima. Possible blood damage due to an incompletely occluding rotor of the hose roller pump can thus be recognized fast and reliably. A mechanical defect of the rotor which is characterized by increased friction of the oscillation or by an oblique roller position can also be detected. For such a mechanical defect also has effects on the time extent of the pressure and can accordingly be detected.

The case is also covered by the invention that a reduced occlusion is recognized in that the time extent of the pressure of a fluid conveyed by means of the hose roller pump has a local minimum.

There is furthermore an increase in safety since the pump function can be directly monitored and no inclusion of the properties of a disposable material, e.g. of a hose, is necessary which is difficult to calculate and is time-dependent, as is the case, for example, in processes which evaluate a pressure signal which is influenced by the hose material used.

Provision can be made that the pressure detection means is arranged downstream of the hose roller pump and upstream of a filter, in particular of a dialyzer, and the pressure profile in this region is a prefilter pressure profile, with the time extent of the pressure detected downstream of the hose roller pump being the prefilter pressure profile, and that the prefilter pressure profile can be continuously detected.

Provision can furthermore be made that a reduced occlusion can be recognized, in particular a premature lifting off of a roller of the hose roller pump is recognized, with reference to an increase in the time interval between the maximum and minimum of the time extent of the pressure.

It is possible that the apparatus has at least one warning means and/or at least one control and/or regulation means and that, in a case in which a reduced occlusion is recognized, at least one alarm can be emitted by means of the warning means and/or the conveying rate can be reduced by means of the control and/or regulation means, in particular the conveying rate for or up to the restoration of a complete occlusion can be reduced automatically or semi-automatically.

Provision can furthermore be made that the monitoring of the time extent of the pressure can be carried out simultaneously with a comparison of the angular position of the hose roller rotor of the hose roller pump.

It is furthermore possible that the apparatus is a component of a blood treatment apparatus and/or is in communication with a blood treatment apparatus and/or is and/or comprises a blood treatment apparatus.

It is also conceivable that at least parts of the apparatus are made as disposables and are in particular integrated into a disposable cassette for use in a blood treatment apparatus in accordance with claim 7. At least one hose part can in particular be arranged in such a cassette for use in the hose roller pump and/or the pressure detection means or at least a part of the pressure detection means.

In a further embodiment of the invention, the apparatus comprises means by means of which the angular position of the rotor of the hose roller pump can be determined.

The angular position of the hose roller rotor of the hose roller pump can, for example, be detected via Hall sensors, optical sensors, angle of rotation potentiometers, etc. and can be transmitted to the monitoring means.

The present invention furthermore relates to a method having the features of claim 10. Provision is accordingly made that a method for monitoring the occlusion of a hose roller pump is carried out such that the hose roller pump has at least one pump bed and at least one hose or hose part inserted or insertable into the pump bed, that the time extent of the pressure of a fluid conveyed by means of the hose roller pump downstream of the hose roller pump is determined and the occlusion of the hose or hose part inserted or insertable into the pump bed is monitored with reference to the time difference and/or the angular difference between at least one maximum and at least one minimum of the determined time extent of the pressure and/or with reference to a local minimum of the determined time extent of the pressure.

Provision can furthermore be made that the time extent of the pressure determined downstream of the hose roller pump is the prefilter pressure profile and that the prefilter pressure profile is continuously detected.

It is furthermore conceivable that a reduced occlusion is recognized, in particular a premature lifting off of a roller of the hose roller pump, is recognized, with reference to an increase in the time interval between the maximum and minimum of the time extent. A lifting of the hose roller off the hose due to a high counterpressure downstream is in particular equivalent to a no longer present occlusion between the hose segment in the pump bed after the occluding roller which occludes toward the inlet and to the outlet of the pump. An abrupt expansion of the pressure thus takes place downstream of the hose roller pump, which can be used for recognizing the change in the occlusion. In normal operation, that is, without premature lifting off of the roller, this expansion always takes place at the same position of the rollers, namely when the one roller exits the pump bed downwardly and can no longer occlude the inserted hose. Just before this point in time or before the corresponding angular position of the hose roller pump, the pressure downstream of the roller pump is at a maximum; briefly thereafter it is at a minimum. A premature lifting off of the roller due to too high a counterpressure can thus be recognized by an early expansion of the after-pump pressure.

It is furthermore conceivable that a reduced occlusion is recognized in that the pressure profile has a local minimum. It has been shown in practice that the prefilter pressure can form a local minimum before the loss of occlusion. A local minimum is in this respect characterized in that the prefilter pressure first drops in the time extent in order to increase again briefly later without the pressure value adopting an absolute minimum. The detection of such a local minimum can be used for recognizing a reduced occlusion.

Provision can furthermore be made that, in a case in which a reduced occlusion is recognized, at least one alarm is emitted and/or a reduction in the conveying rate is carried out, in particular a reduction in the conveying rate for or up to the restoration of a complete occlusion is carried out automatically or semi-automatically.

It is further possible that the monitoring of the time extent of the pressure takes place together with a comparison of the angular position of the hose roller rotor of the hose roller pump.

As stated above, suitable means can be provided by means of which the angular position of the hose roller rotor is detected. These means can, for example, comprise Hall sensors, optical sensors, angle of rotation potentiometers, etc. These means are preferably made so that they transmit the detected angular position in the form of a signal to the monitoring means or are in communication with such transmission means.

It is furthermore advantageously conceivable that the method is carried out with at least one apparatus in accordance with one of the claims 1 to 9.

The invention furthermore relates to a use of an apparatus having a hose roller pump having the features of claim 16. Provision is accordingly made that an apparatus in accordance with claims 1 to 9 is used for the performance of the method in accordance with claims 10 to 15 and/or in a blood treatment apparatus, in particular in a hemodialysis machine.

The present invention furthermore relates to a blood treatment apparatus having the features of claim 17. Provision is accordingly made that a blood treatment device, in particular a dialysis machine, is provided with at least one apparatus in accordance with one of the claims 1 to 9.

The present invention furthermore relates to the use of a blood treatment apparatus having the features of claim 18. Provision is accordingly made that a blood treatment apparatus, in particular a blood treatment apparatus in accordance with claim 17 is used for performing the method in accordance with claims 10 to 15.

The present invention furthermore relates to a disposable having the features of claim 19. Provision is accordingly made that a disposable, in particular a disposable hose kit, is provided for use in an apparatus in accordance with one of the claims 1 to 9.

The present invention furthermore relates to a cassette having the features of claim 20. Provision is accordingly made that a cassette, in particular a disposable cassette, is provided for use in and/or with an apparatus in accordance with one of the claims 1 to 9, with the cassette having at least one hose part for insertion into a part of the hose roller pump and/or with the pressure detection means and/or a part of the pressure detection means being arranged in the cassette.

Further details and advantages of the invention will now be explained in more detail with reference to an embodiment shown in the drawing. There are shown:

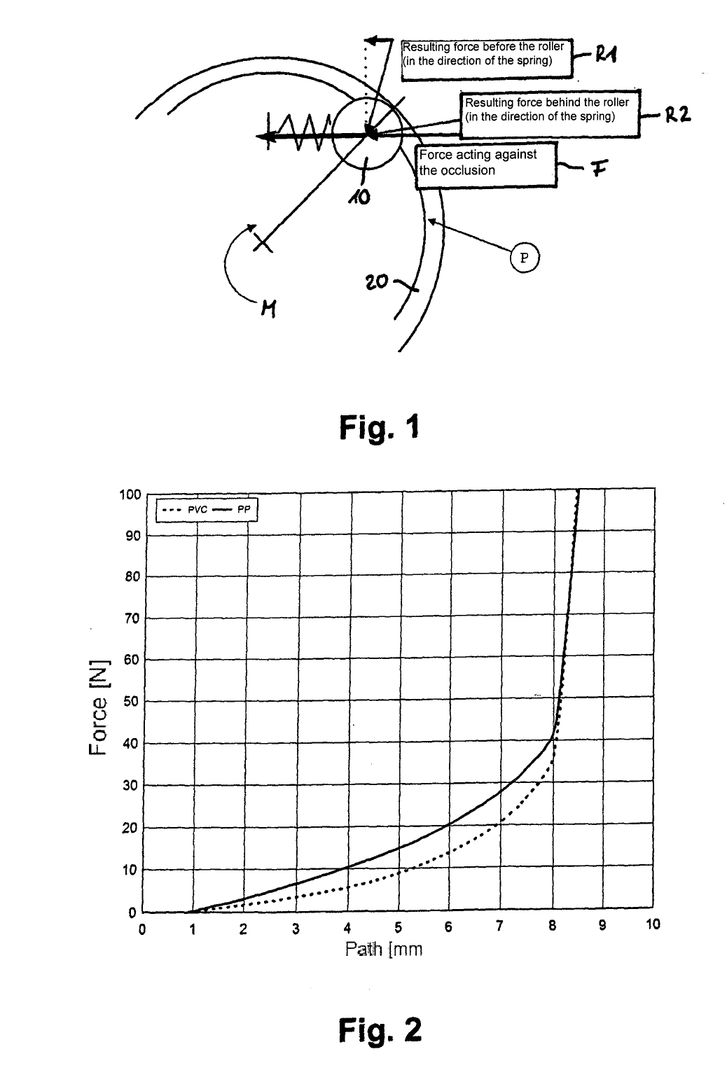

FIG. 1: a schematic observation of the forces and pressures applied in a hose roller pump;

FIG. 2: a diagram relating to the force/path characteristics of hose materials;

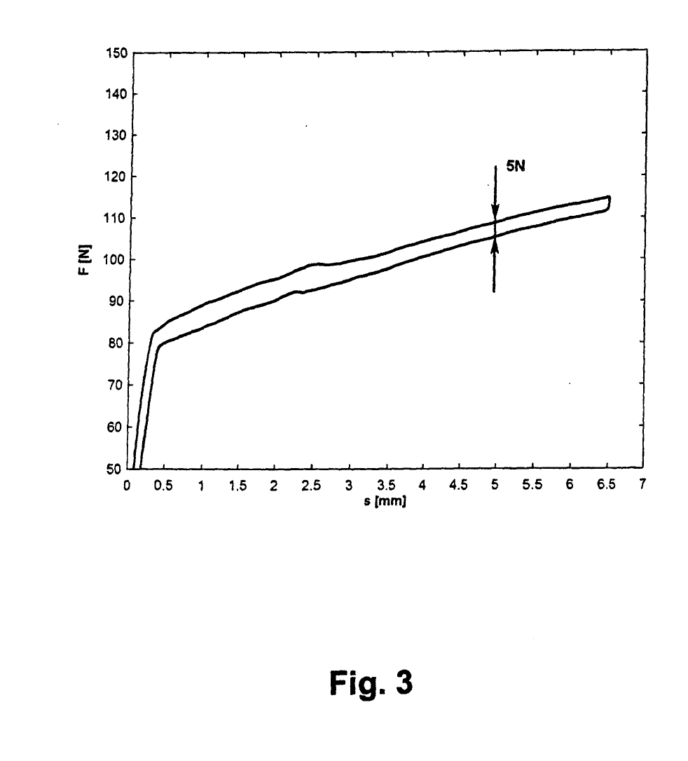

FIG. 3: the spring characteristic of a rotor of a hose roller pump; and

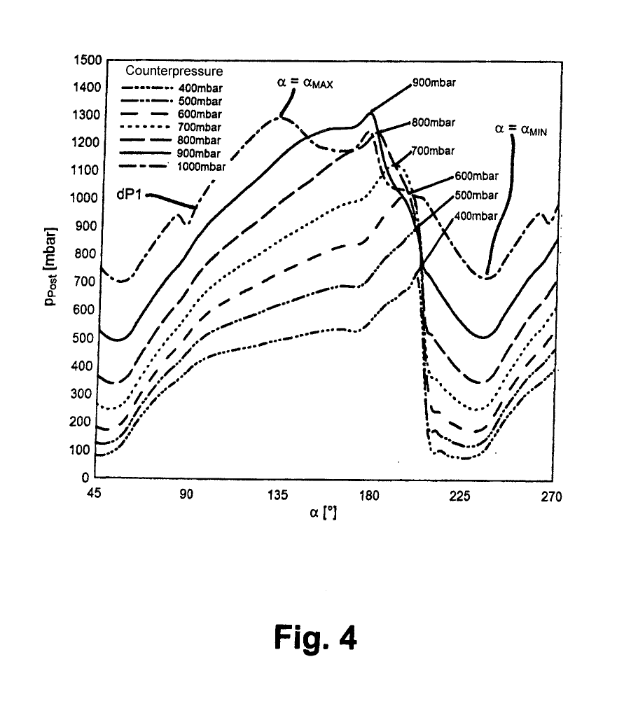

FIG. 4: a diagram relating to the prefilter pressure profile with different characteristics with increasing counterpressure.

The method in accordance with the invention is advantageously performed as follows on an apparatus not shown in any more detail:

The prefilter pressure signal is detected in a time resolution which is as high as possible. The course of the pressure signal in the working area is largely reproducible, i.e. at the same angle of rotation α (cf. also FIG. 4), the same pressure can also be measured as a rule.

The shape of the pressure profile is above all determined by the pump principle. The conveying (increase in the pressure or pressure constant) and the disengagement of the roller (abrupt pressure drop) is characteristically recognizable in the pressure profile at the prefilter pressure sensor.

The angular position of the lifting off of the roller is dependent on the shape of the pump bed and on the (resulting) force with which the hose is compressed.

The pump bed is shaped so that the roller disengagement is introduced from a defined angle onward. The roller follows the run-out chamfer for so long until it has reached its end position and the rocker at the inner abutment of the rotor is prevented from further outward movement. The force results, on the one hand, from the spring force with which the roller presses against the hose and, on the other hand, from the pressure in the hose which acts against this spring force and from the restoring force of the hose material.

FIG. 1 schematically shows the primarily relevant applied forces or parameters, namely in detail the resulting force R1 before the roller 10 which acts in the direction of the spring; the resulting force R2 behind the roller in the direction which acts in the direction of the spring; the force F which acts against the occlusion and which is the restoring force F of the material of the hose 20; the torque M; and the prefilter pressure P.

In particular the restoring force of the hose material changes greatly in the course of the treatment.

In the graphs in accordance with FIG. 2 the force/path characteristics of some hose materials are shown, namely of PVC and PP. From a path of approximately 8 mm, the hoses are practically completely occluded. The hose diameter here amounts to 12 mm and the wall thickness to 2 mm.

To occlude the hoses completely, a force in the range of 40 N is necessary here. A complete occlusion of the hose is usually only reached above this force.

If the spring characteristic (hysteresis measurement at environmental temperature) of a “normal” rotor in accordance with FIG. 3 is observed, it can be recognized that the restoring force of the hose of approximately 40 N is approximately half as much as the spring force of the rotor at the operating point (approx. 90 N) and thus has a substantial influence on the pressure from which onward a complete occlusion is no longer present.

The prefilter pressure profile is continuously detected during the dialysis. It can be analyzed as a curve profile or be compared with the angular position of the blood pump rotor for increasing the tolerance with respect to changes in the pump hose material, medium, etc.

If the maximum of the pressure profile migrates too far “to the left” (cf. FIG. 4) or if the time interval between the maximum and the minimum considerably increases, the roller prematurely lifts off and there is the risk of blood damage. A reduction in the conveying rate for restoring a complete occlusion or a premature alarm reaction can be initiated.

In FIG. 4, the pressure profiles, relative to the rotor position, were entered at different counterpressures. For this purpose, a rotor was used having two oppositely disposed rollers with reduced spring force and the flow behind the pump is different from 0 ml/min. The rotor of the hose roller pump with which the diagram in accordance with FIG. 4 was determined is a rotor having two oppositely disposed rollers. For example, at an angular position of α=0° and α=180°, there is still full occlusion by at least one roller, with this roller exiting the pump bed via the run-out chamfer as a consequence of the further rotational movement.

It is, however, generally possible to make the hose roller pump as desired, in particular with respect to the number of rollers and to the pump bed design.

It can be recognized that, as the counterpressure increases, here from approx. 700 mbar onward, the maximum of the prefilter pressure profile migrates “to the left”, i.e. α_{MAX} occurs at an earlier time or at an earlier angular position during the rotation of the rotor. The angular position α_{MIN} of the minimum, in contrast, remains practically unchanged.

The uppermost curve dP1 thus shows an almost completely lifted off blood pump rotor and differs considerably from the other curves.

1. An apparatus having at least one hose roller pump, wherein the hose roller pump has at least one pump bed and at least one hose or hose part inserted or insertable into the pump bed, and having at least one pressure detection means which is arranged downstream of the hose roller pump and by means of which the time extent of the pressure of a fluid conveyed by means of the hose roller pump can be determined, and having at least one monitoring means by means of which the occlusion of the hose or hose part inserted or insertable into the pump bed can be monitored with reference to the time difference and/or to the angular difference between at least one maximum and at least one minimum of the determined time extent of the pressure and/or with reference to a local minimum of the determined time extent of the pressure.

2. An apparatus in accordance with claim 1, characterized in that the pressure detection means is arranged downstream of the hose roller pump and upstream of a filter, in particular of a dialyzer, and the pressure profile in this region is a prefilter pressure profile, with the time extent of the pressure detected downstream of the hose roller pump being the prefilter pressure profile; and in that the prefilter pressure profile can be continuously detected.

3. An apparatus in accordance with claim 1, characterized in that a reduced occlusion can be recognized, in particular a premature lifting off of a roller of the hose roller pump is recognized, with reference to an increase in the time interval between the maximum and minimum of the time extent of the pressure.

4. An apparatus in accordance with claim 1, characterized in that a reduced occlusion can be recognized, in particular a premature lifting off of a roller of the hose roller pump is recognized, with reference to the occurrence of a local minimum of the determined time extent of the pressure.

5. An apparatus in accordance with claim 3, characterized in that the apparatus has at least one warning means and/or at least one control and/or regulation means; and in that, in a case in which a reduced occlusion is recognized, at least one alarm can be emitted by means of the warning means and/or the conveying rate can be reduced by means of the control and/or regulation means, in particular the conveying rate for or up to the restoration of a complete occlusion can be reduced automatically or semi-automatically.

6. An apparatus in accordance with claim 1, characterized in that the monitoring of the time extent of the pressure can be carried out together with a comparison of the angular position of the hose roller rotor of the hose roller pump.

7. An apparatus in accordance with claim 1, one characterized in that the apparatus is a component of a blood treatment apparatus and/or is in communication with a blood treatment apparatus and/or is and/or includes a blood treatment apparatus.

8. An apparatus in accordance with claim 1, characterized in that at least parts of the apparatus are made as disposables and are in particular integrated into a disposable cassette for insertion into a component of a blood treatment apparatus and/or is in communication with a blood treatment apparatus and/or is and/or includes a blood treatment apparatus.

9. An apparatus in accordance with claim 1, characterized in that the apparatus has means by means of which the angular position of the rotor of the hose roller pump can be detected.

10. A method for monitoring the occlusion of a hose roller pump is carried out such that the hose roller pump has at least one pump bed and at least one hose or hose part inserted or insertable into the pump bed, wherein the time extent of the pressure of a fluid conveyed by means of the hose roller pump downstream of the hose roller pump is determined and the occlusion of the hose or hose part inserted or insertable into the pump bed is monitored with reference to the time difference and/or the angular difference between at least one maximum and at least one minimum of the determined time extent of the pressure and/or with reference to a local minimum of the determined time extent of the pressure.

11. A method in accordance with claim 10, characterized in that the time extent of the pressure detected downstream of the hose roller pump is the prefilter pressure profile; and in that the prefilter pressure profile is continuously detected.

12. A method in accordance with claim 10, characterized in that a reduced occlusion is recognized, in particular a premature lifting off of a roller of the hose roller pump is recognized, with reference to an increase in the time interval between the maximum and minimum of the time extent of the pressure.

13. A method in accordance with claim 12, characterized in that, in a case in which a reduced occlusion is recognized, at least one alarm is emitted and/or a reduction in the conveying rate is carried out, in particular a reduction in the conveying rate for or up to the restoration of a complete occlusion is carried out automatically or semi-automatically.

14. A method in accordance with claim 10, characterized in that the monitoring of the time extent of the pressure takes place together with a comparison of the angular position of the hose roller rotor of the hose roller pump.

15. A method for monitoring the occlusion of a hose roller pump carried out such that the hose roller pump has at least one pump bed and at least one hose or hose part inserted or insertable into the pump bed, wherein the time extent of the pressure of a fluid conveyed by means of the hose roller pump downstream of the hose roller pump is determined and the occlusion of the hose or hose part inserted or insertable into the pump bed is monitored with reference to the time difference and/or the angular difference between at least one maximum and at least one minimum of the determined time extent of the pressure and/or with reference to a local minimum of the determined time extent of the pressure, characterized in that the method is carried out using at least one apparatus in accordance with claim 1.

16. Use of an apparatus having at least one hose roller pump, wherein the hose roller pump has at least one pump bed and at least one hose or hose part inserted or insertable into the pump bed, and having at least one pressure detection means which is arranged downstream of the hose roller pump and by means of which the time extent of the pressure of a fluid conveyed by means of the hose roller pump can be determined, and having at least one monitoring means by means of which the occlusion of the hose or hose part inserted or insertable into the pump bed can be monitored with reference to the time difference and/or to the angular difference between at least one maximum and at least one minimum of the determined time extent of the pressure and/or with reference to a local minimum of the determined time extent of the pressure for carrying out of the method in accordance with claim 10 and/or in a blood treatment apparatus, in particular in a hemodialysis machine.

17. A blood treatment apparatus, in particular a hemodialysis machine, having at least one apparatus in accordance with claim 1.

18. Use of a blood treatment apparatus, in particular, a hemodialysis machine, having at least one apparatus having at least one hose roller pump, wherein the hose roller pump has at least one pump bed and at least one hose or hose part inserted or insertable into the pump bed, and having at least one pressure detection means which is arranged downstream of the hose roller pump and by means of which the time extent of the pressure of a fluid conveyed by means of the hose roller pump can be determined, and having at least one monitoring means by means of which the occlusion of the hose or hose part inserted or insertable into the pump bed can be monitored with reference to the time difference and/or to the angular difference between at least one maximum and at least one minimum of the determined time extent of the pressure and/or with reference to a local minimum of the determined time extent of the pressure, for carrying out the method in accordance with claim 10.

19. A disposable, in particular a disposable hose kit, for use in an apparatus in accordance with claim 1.

20. A cassette, in particular a disposable cassette for use in and/or with an apparatus in accordance with claim 1, wherein the cassette has at least one hose part for insertion into a part of the hose roller pump and/or wherein the pressure detection means and/or a part of the pressure detection means is arranged in the cassette.
